(19) United States
(12) Patent Application Publication
(10) Pub. No.: US 2024/0037748 A1
(43) Pub. Date: Feb. 1, 2024

(54) METHODS FOR CLASSIFICATION OF LESIONS AND FOR PREDICTING LESION DEVELOPMENT

(71) Applicant: BIOGEN MA INC., Cambridge, MA (US)

(72) Inventors: Bastien CABA, Vancouver (CA); Dawei LIU, Sharon, MA (US); Aurélien LOMBARD, Verton (FR); Alexandre CAFARO, Montrouge (FR); Elizabeth FISHER, Lexington, MA (US); Arie Rudolf GAFSON, Unterageri (CH); Nikos PARAGIOS, Paris (FR); Shibeshih Mitiku BELACHEW, Zürich (CH); Xiaotang Phoebe JIANG, Boston, MA (US)

(73) Assignee: BIOGEN MA INC., Cambridge, MA (US)

(21) Appl. No.: 18/483,571

(22) Filed: Oct. 10, 2023

Related U.S. Application Data
(63) Continuation of application No. PCT/US2022/024450, filed on Apr. 12, 2022.

(30) Foreign Application Priority Data
Apr. 13, 2021 (FR) 2103793

(51) Int. Cl. G06T 7/00; G06T 7/11; A61B 5/00; A61B 5/055; G16H 30/40

(52) U.S. Cl. CPC G06T 7/0016; G06T 7/11; A61B 5/0042; A61B 5/055; A61B 5/7267; G16H 30/40; G06T 2207/10088; G06T 2207/30016; G06T 2207/30096; G06T 2207/20081; A61B 2576/026

(57) ABSTRACT

Disclosed are systems and methods for classifying brain lesions based on single point in time imaging, methods for training a machine learning model for classifying brain lesions, and a method of predicting formation of brain lesions based on single point in time imaging. A method of classifying brain lesions based on single point in time imaging can include; accessing patient image data from a single point in time; providing the patient image data as an input to a brain lesion classification model; generating a classification for each of one or more lesions identified in the patient image data; and providing the classification for each of the one or more lesions for display on one or more display devices; wherein the brain lesion classification model is trained using subject image data for a plurality of subjects, the subject image data being captured at two or more points in time.

### CROSS-REFERENCE TO RELATED APPLICATIONS

This application claims priority to French Provisional Application No. 2103793 filed Apr. 13, 2021, the entire disclosure of which is hereby incorporated herein by reference in its entirety.

### TECHNICAL FIELD

Various aspects of the disclosure relate generally to systems and methods for machine-learning-assisted lesion classification and progression prediction. According to examples, the disclosure relates to methods for analyzing patient images (e.g., magnetic resonance imaging scans), identifying biomarkers, which may include first—as well as higher-order textural features, related to the activity, stage (e.g., new or old) and/or likely progression of a lesion (e.g., a multiple sclerosis lesion), and determining characteristics that may be beneficial in diagnosis, monitoring, prognosis and/or treatment, including, for example, of multiple sclerosis.

### BACKGROUND

Multiple sclerosis (“MS”) is a chronic disease of the central nervous system, which affects the brain, spinal cord, and optic nerves, among other things. The disease impacts the patient as the immune system attacks healthy tissue in the central nervous system, resulting in damage to the myelin that surrounds the nerve fibers as well as damage to the nerves themselves. This damage, often appearing as lesions in the brain, disrupts the transmission of nerve signals within the brain, as well as between the brain and spinal cord and other parts of the body. Over two million people worldwide have multiple sclerosis, the cause is unknown and the disease is currently considered incurable.

However, there exist treatments that act to slow the progression of the damage, and reduce the amount and progression of lesions in the brain. In order to appropriately monitor and treat MS, healthcare professionals have a need to understand not only the number, the location and the volumetric aspect of lesions that are present in the patient, but which of those lesions are active at any given time and whether they are ‘new’ or ‘old’ (i.e., were visible on a previous scan).

Conventional methods of detecting and classifying MS lesions rely on the eyes and judgment of a radiologist. These methods are based on the radiologist's review of multiple magnetic resonance imaging (“MRI”) scans conducted using different methods, and often rely on conventional and contrast enhanced MRI scans captured at different points in time.

MRI scanners use strong magnetic fields and radio waves to produce images that correspond to the properties of the tissues in the human body. However, there are different methodologies (known as sequences), that produce images reflecting different tissue properties. For example, a T1-weighted scan measures a property called spin-lattice relaxation by using a short repetition time as well as a short echo time. The resulting images will show a lower signal (darker color) for tissues and areas with a high water content, and a higher signal (brighter color) for fatty tissues. On the other hand, a T2-weighted scan measures a property called spin-spin relaxation by using longer repetition times and longer echo times. Images that result from a scan performed with T2-weighting will show a higher signal for areas of higher water content, and will show fatty tissue with a lower signal.

Another difference between the sequences is how they respond to contrast agents, such as gadolinium. Gadolinium is known as a paramagnetic contrast agent that increases the signal measured during a T1-weighted scan, but does not increase the signal for T2-weighted scans. In practice, gadolinium and other paramagnetic contrast agents, are visible as they cross the blood-brain barrier and therefore highlight areas where the blood-brain barrier is compromised, such as areas of active inflammation.

As it relates to diagnosing and monitoring MS, various sequences can be used, with each potentially indicating different aspects of the disease and damage. For example:
T1-weighted scans conducted without paramagnetic contrast agents may show dark areas that may indicate areas of permanent neural tissue damage.
T1-weighted scans conducted after intravenous administration of paramagnetic contrast agents (such as gadolinium) may indicate areas of acute inflammation as brightly enhanced in comparison to locations where the blood-brain barrier is intact.
T2-weighted scans will show regions of brighter signal (hyperintensities) where the myelin that typically covers the nerves in brain white matter has been stripped away. These images can indicate the presence of an MS lesion, but it does not distinguish between the acute lesions and chronic lesions that are not presently inflamed.
   When images from these sequences are viewed together, a radiologist is able to identify the total lesion burden from the T2-weighted scan, with the lesions that also are enhanced by gadolinium on the T1-weighted images being considered acute.

However, these conventional detection methods may underestimate acute MS pathology, due to the transient nature of blood-brain barrier disruption and gadolinium enhancement that indicate an acute MS lesion (a new lesion will enhance on average for 1.5 to 3 weeks). Furthermore, the contrast agent, gadolinium, used during these scans for acute MS lesion detection may pose some risk to the patient (e.g., the patient's renal system) or may result in deposits of contrast agent forming in the tissues of the patient, including the brain. The recent acute MS lesions can also be detected by comparing two T2-weighted scans at different points in time (e.g., 3-12 months apart); a recent acute MS lesion will then be defined by the identification of a new T2 hyperintense lesion on the second scan in reference to the prior acquisition. As a result, conventional detection methods may be complex and expensive due to the need to conduct multiple scans at multiple points in time, they can slow down decision making in clinic because they rely on longitudinal scans and they are associated with potential risks posed by frequent use of gadolinium contrast agents.

The present disclosure is directed to methods and systems focused on addressing one or more of these above-referenced challenges or other challenges in the art.

### SUMMARY

Aspects of the disclosure relate to, among other things, systems and methods for machine-learning-assisted lesion classification and progression/appearance prediction. In embodiments, methods for analyzing patient images (e.g., MRI scans), may proceed by identifying, in patient MRI data, biomarkers that can include first-, second- and higher-order features related to the activity, temporal status (e.g., acute or chronic) and/or likely progression of a lesion (e.g., an MS lesion, chronic active or inactive, expanding/evolving or non-expanding/evolving, and/or harboring the specific pattern of a lesion subtype), and determining characteristics that may be beneficial in the diagnosis, monitoring, and/or treatment, for example, of MS. Each of the aspects disclosed herein may include one or more of the features described in connection with any of the other disclosed aspects.

In one aspect, an exemplary method of classifying brain lesions based on single point in time imaging can include; accessing, by a system server, patient image data from a single point in time; providing, by the system server, the patient image data as an input to a brain lesion classification model; generating, by the brain lesion classification model, a classification for each of one or more lesions identified in the patient image data; and providing the classification for each of the one or more lesions for display on one or more display devices; wherein the brain lesion classification model is trained using subject image data for a plurality of subjects, the subject image data for each of the plurality of subjects being captured at two or more points in time.

In another aspect, an exemplary system for classifying brain lesions based on single point in time imaging can include a memory configured to store instructions; and a processor operatively connected to the memory and configured to execute the instructions to perform a process. The process can include: accessing, by a system server, patient image data from a single point in time; providing, by the system server, the patient image data as an input to a brain lesion classification model; generating, by the brain lesion classification model, a classification for each of one or more lesions identified in the patient image data; and providing the classification for each of the one or more lesions for display on one or more display devices; wherein the brain lesion classification model is trained using subject image data for a plurality of subjects, the subject image data for each of the plurality of subjects being captured at two or more points in time.

In a further aspect, an exemplary method for training a machine-learning model for classifying brain lesions can include: obtaining, via a system server, first training data that includes information for a plurality of subjects including image scan data for each subject captured at two or more points in time and obtaining, via the system server, second training data that includes classification information for one or more brain lesions present in the image scan data, wherein the classification information for the one or more brain lesions present in the image scan data is indicative of a classification of the one or more brain lesions as being acute or chronic. The method can further include extracting, from the first training data, one or more patches representing one or more brain lesions; extracting, from each of the one or more patches representing one or more brain lesions, a plurality of biomarkers; and determining, within the plurality of biomarkers, a subset of biomarkers relevant to the classification of the one or more brain lesions as correlated with the second training data.

In an additional aspect, an exemplary method of predicting a formation of brain lesions based on single point in time imaging can include: accessing, by a system server, patient image data from a single point in time; providing, by the system server, the patient image data as an input to a brain lesion prediction model; generating, by the brain lesion prediction model, a prediction for the patient image data, the prediction including an indication of a likelihood of a future lesion forming; and providing the prediction for the patient image data for display on one or more display devices; wherein the brain lesion prediction model is trained using subject image data for a plurality of subjects, the subject image data for each of the plurality of subjects being captured at two or more points in time.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated herein and constitute a part of this specification, illustrate exemplary aspects of the disclosure and, together with the description, explain the principles of the disclosure.

FIG. 1 depicts a flowchart of an exemplary method of lesion classification, according to one or more embodiments.

FIG. 2 depicts a flowchart of an exemplary method for identifying biomarkers for the classification of brain lesions, according to one or more embodiments.

FIG. 3 depicts a process used to define lesion masks, according to one or more embodiments.

FIGS. 4A-C depict axial, coronal, and sagittal views of acute and chronic lesion masks, according to one or more embodiments.

FIG. 5 depicts an exemplary lesion inpainting model architecture, according to one or more embodiments.

FIGS. 6A-C depict an inpainting process including original, masked, and inpainted images, according to one or more embodiments.

FIGS. 7A-F depict a patch extraction procedure, according to one or more embodiments.

FIG. 8A-E depicts a process for segmenting regions of interest within patches, according to one or more embodiments.

FIG. 9 depicts a classification and feature selection pipeline, according to one or more embodiments.

FIG. 10 depicts a flowchart of an exemplary method of lesion prediction, according to one or more embodiments.

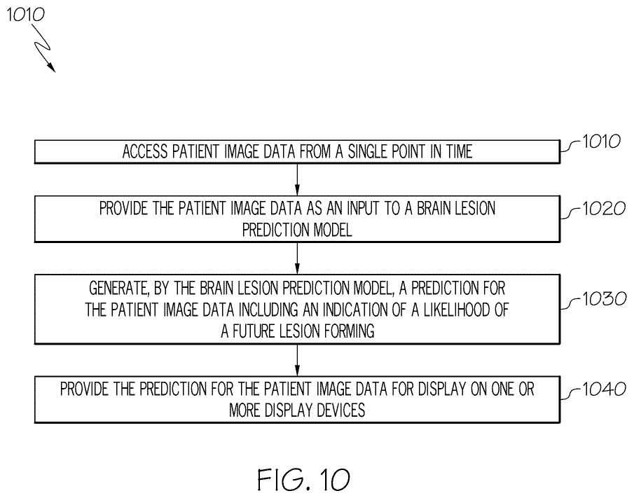

FIG. 11 depicts an example of a computing device, according to one or more embodiments.

### DETAILED DESCRIPTION

Embodiments of this disclosure relate to analysis of MRI images of MS patients to enable conclusions to be drawn that are not possible or practical with the eyes of a radiologist alone.

By collecting and analyzing sets of MRIs from MS patients using machine learning techniques, the disclosed methods distinguish between acute and chronic lesions using only T1-weighted and T2-weighted scans conducted without gadolinium enhancement and taken at a single point in time. Methods according to the present disclosure may identify features present in the unenhanced T1-weighted and T2-weighted MRI data that correlate with the acute nature of a lesion, as detected by a traditional gadolinium-enhanced sequence, or via a comparison of multiple longitudinal T2-weighted MRI scans. As a result, the disclosed methods may accurately and reproducibly discriminate between acute and chronic lesions in a manner not presently practical for a radiologist alone.

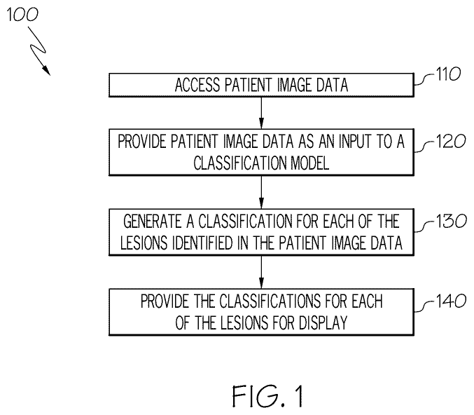

Methods in accordance with the present disclosure, such as exemplary method 100 shown in FIG. 1, may begin at step 110 with accessing patient image data to be classified. This patient data may include, for example, MR images and/or data collected at a single point in time, such as on the same day or session in the MRI machine. These images can include, for example, T1- and T2-weighted MR images that do not include the administration of a paramagnetic contrast agent such as gadolinium.

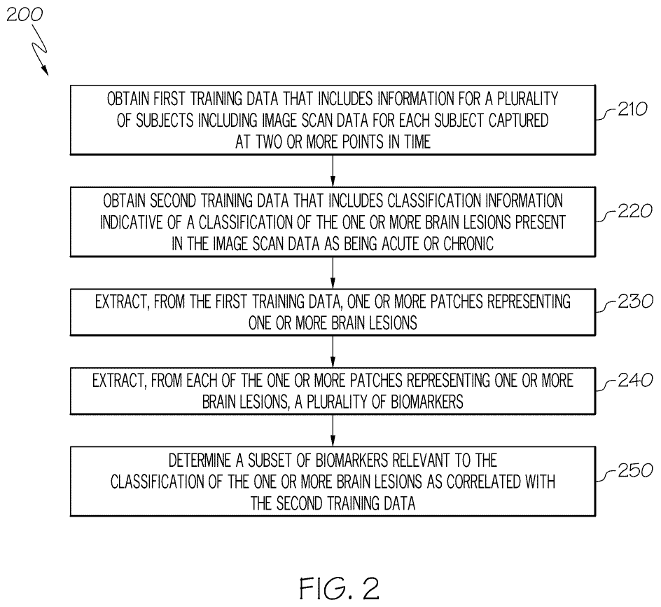

At step 120, the patient image data can then be provided as an input to a classification model. An exemplary method 200 for identifying biomarkers for the classification of brain lesions to generate a classification model is discussed with respect to FIG. 2.

Method 200 can begin, at step 210, by obtaining a first set of training data that includes a collection of clinical data, for example MRI images from subjects diagnosed with MS. The first set of training data may include feature data, for example, MR images collected at two or more points in time that are, for example, at least over one week apart, such as about 24 weeks to 36 weeks apart. The timing between the two or more points in time includes a time period relevant to demonstrate an anatomical change, should one occur, such as the growth of a lesion and/or tumor. The first set of training data may include scans conducted with and/or without a paramagnetic contrast agent, and conducted using one or more scan sequences, such as T1- and T2-weighted sequences.

At step 220, the system can obtain a second set of training data, which can include label data, for example, a set of labels associated with the first set of training data including classification information for brain lesions identified in the collection of clinical data. The labels can include, for example, ground truth segmentations of acute and chronic MS lesions. This data may be generated by, for example, individuals such as radiologists, groups/panels of patient care providers, or another relevant source of actual clinical lesion diagnosis or determinations regarding the subject image data.

At step 230, patches may be extracted from the first set of clinical training data. However, prior to patch extraction, the first set of training data may be curated and normalized, to produce a representational data set where irrelevant sources of variability are eliminated whereas the variability associated with differences between the observed lesion classes is conserved. In the context of multiple sclerosis lesion classification, this normalization may be applied to account for disease-independent anatomical differences observed across patients, differences in MR image acquisition parameters, or significant distributional imbalance that may be present in the collection of images, particularly as between acute and chronic lesions intra- and inter-patient. By looking into the distribution of lesions once brought to a common space from subjects coming from multiple data sets, the collection may be normalized to exhibit intra/inter-patient and intra/inter-clinical study representation that appears more consistent and conserves both geometrical and appearance variability across data samples. According to the disclosed methods, aggregation of data coming from multiple studies can be done suitably on the basis of statistical and machine learning principles leading to a task-specific sampling and normalization strategy. Such a strategy is modular, scalable, and task-specific allowing the method to decipher appropriate information related to the classification task. This representation may be used to create a robust training set that encompasses the observed location, disease-extent, and imaging characteristic variability across a particular disease group (e.g., MS).

The machine learning training data set should account for the receipt of subject data across different imaging devices. For example, subject data may be received from imaging devices using different settings, from different vendors, and/or having magnets with different field strengths and specifications. The machine learning algorithm should apply across these different machines. The disclosed methods, as part of step 120, may apply various normalization approaches, including, for example:
full-brain normalization and bias-field correction (e.g., on the basis of the whole brain distribution, the z-scoring principle is used to rescale the values of each patient to a distribution with zero mean, unit variance);
gray/white matter normalization (e.g., on the basis of the gray/white matter signal distribution, the min-max principle is used to rescale the values of each subject to the [0,1] interval); and
normalization via distribution mapping to a common reference distribution (e.g., on the basis of the whole brain distribution, piecewise-linear histogram matching is performed between each subject and the reference histogram).
   Having been normalized, the first set of training data can be further processed and analyzed.

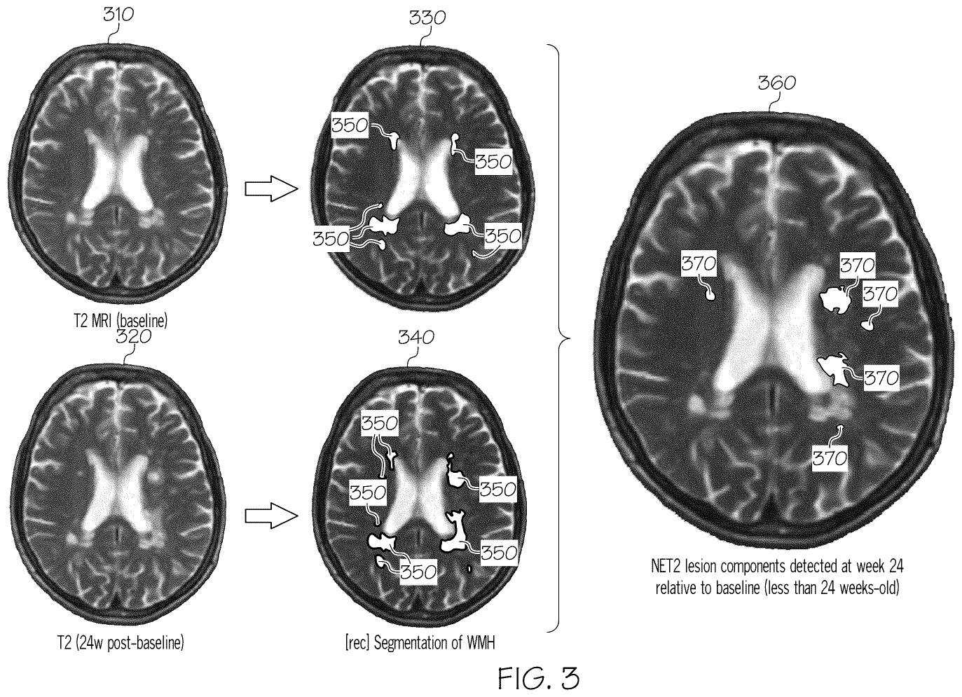

FIG. 3 depicts an exemplary process for defining masks corresponding to one or more lesions present in the image. The lesions may be identified on the image as regions of high intensity signal within the white matter known as white matter hyperintensities (WMH). These WMH regions, as identified in baseline scan 310 and post-baseline scan 320, can be segmented in each longitudinal T2-weighted MRI scan, for example, as indicated by segmented baseline scan 330 and segmented post-baseline scan 340. The regions of WMH 350 identified in segmented scans 330 and 340 can be compared, such that new WMHs 350 detected in segmented scan 340 relative to a prior reference scan 330 can be identified. These new or substantially enlarging T2 (NET2) lesions can be represented as a NET2 mask, which is constructed as the set of voxels which are labeled as WMH at that timepoint t and were not labeled as WMH in a previous timepoint t−1, for example, acquired at most 24 weeks prior to t. For example, composite scan 360 indicates several acute lesion components 370 as the WMH regions 350 that appear on post baseline scan 320 that were not present in baseline scan 310. In some embodiments, other types of masks may be defined, such as slowly-expanding lesion (“SEL”) masks defined as contiguous regions of pre-existing T2 lesion showing gradual concentric expansion sustained over a period of, for example, about 1 to 2 years.

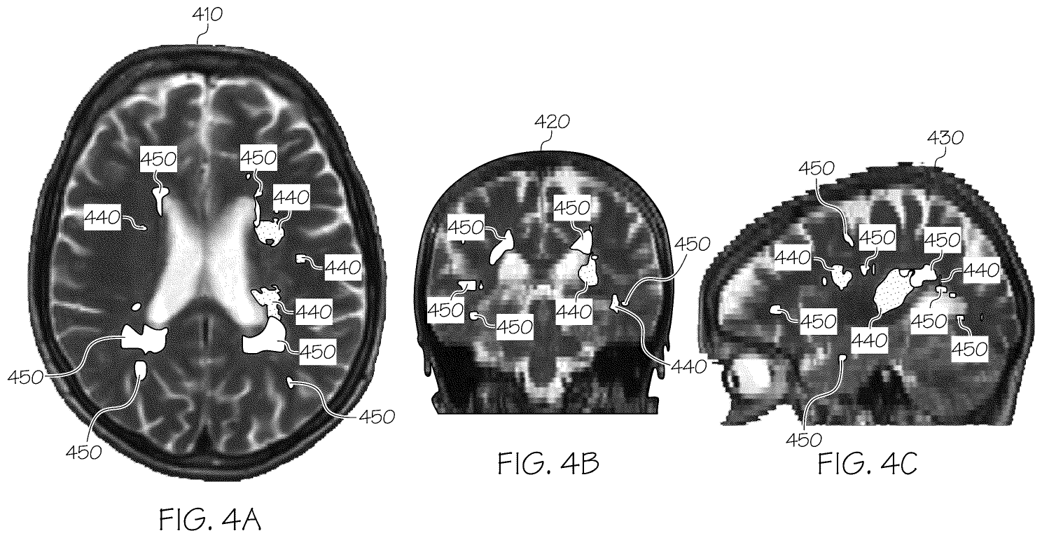

Once the lesion masks are identified and segmented, FIGS. 4A-C illustrate axial (410), coronal (420) and sagittal (430) views of a T2-weighted MRI showing the acute 440 and chronic 450 ground truth segmentation maps. From the segmented and masked images, different approaches may be used to extract imaging biomarker features depicting variability across chronic and acute lesions. These imaging biomarker features might be originated from each view of the original lesion-present image or an artificially generated lesion-free image or from both. The normalized data set may then be used as a training data set for a machine learning feature selection pipeline. The machine learning pipeline may in turn be able to adjust the combination/recovery of biomarker features such that they are able to cover an entire spectrum of visual appearances associated with specific lesion types, while eliminating non-discriminative features equally expressed across all lesion types.

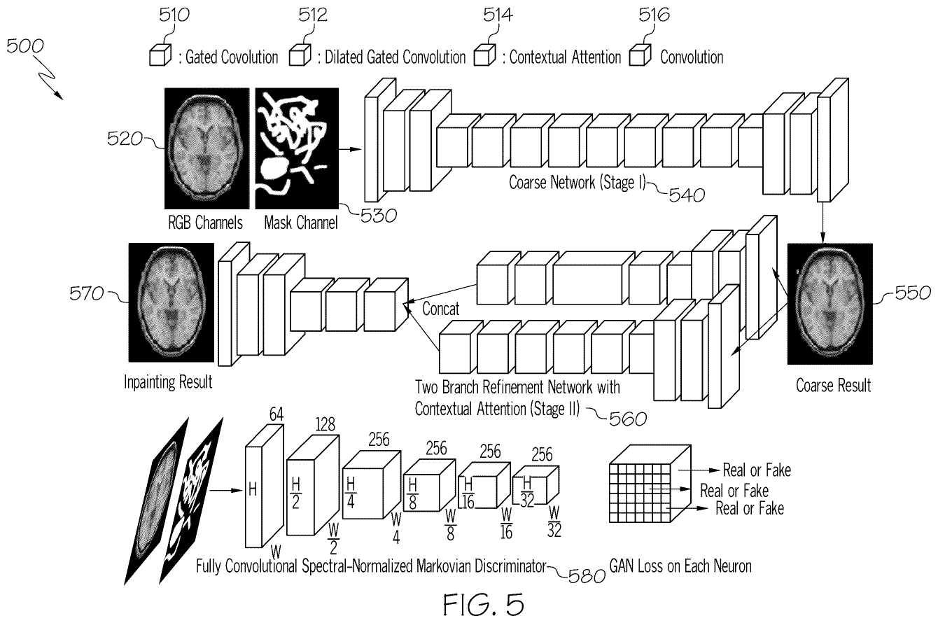

In some applications of the disclosed methods, image synthesis/inpainting techniques may be applied to the first training dataset in order to supplement the training data with additional examples of lesion-free images. In the context of the lesion classification, information relevant to the lesion-free state of a patient may be useful in the assessment of lesion progression (e.g., chronic active or inactive, expanding/evolving or non-expanding/evolving, and/or harboring the specific pattern of a lesion subtype). However, this information is not often available in the context of the clinical trial data adapted into the training data set, and is even less likely to be available in a clinical setting. In order to overcome this, a machine learning or artificial intelligence (AI)-based solution may be employed to generate lesion-free brain content that reproduces the most likely healthy tissue appearance. An example of an inpainting model architecture 500 for generating synthetic lesion-free images is depicted in FIG. 5.

Inpainting model architecture 500 may be based on, for example, a generative adversarial network (GAN) architecture that can be adapted to allow for a multi-view framework to support 3D inpainting. Architecture 500 can include components including: gated convolution 510, dilated gated convolution 512, contextual attention 514, and convolution 516. For example, gated convolution 510 can restrict the spatial region to which the filter has access, while dilated gated convolution 512 can artificially create gaps between its kernel elements, such as to cover a larger spatial extent. In some embodiments, contextual attention 514 can allow the network to focus on specific regions proximate the area to be inpainted, as these regions may contain information that can be used to guide the inpainting process.

As illustrated, one or more channels of data, such as an image channel 520 and mask channel 530, can be fed into architecture 500. Image channel 520 can be one or more images and/or image data combined across different imaging sequences, such as T1-weighted and T2-weighted MR images. Model architecture 500 can be composed of two stacked encoder-decoder generator blocks referred to as the coarse network 540 and the refinement network 560. Coarse network 540 can output a coarse result 550, which then may serve as the input to refinement network 560. These blocks may implement gated convolutions 510 to restrict the encoding-decoding process to information contained outside of the region to be inpainted. The refinement branch can include a contextual attention module 514, such as a recursive self-attention module, to guide the encoding process. Refinement network 560 may then output the inpainting result 570.

The inpainting model can be optimized via minimization of an objective function which may be formulated as a linear combination of loss terms which may include, for example, the L1 distance between the output of the coarse network 540 and the ground truth training image, the L1 distance between the output of the refinement network and the ground truth image, and/or a discriminator loss computed via discrimination block 580. The discriminator loss may be defined, for example, as a fully convolutional Spectral-Normalized Markovian Discriminator. In some embodiments, the convolutions may be standard convolutions, and the output of discriminator 580 may be a scalar number. In some embodiments, discriminator 580 is trained to discriminate real images (taken from the first set of clinical training data) from fake images (generated by the refinement network). The generator can compete with discriminator 580 and attempts to generate artificial images that discriminator 580 assesses as real images. Discriminator 580 may estimate the probability that a given image is real (“D(x)”), such that the output of the GAN loss on each neuron is D(x). Because a well-trained generator can be better able to fool discriminator 580 into thinking the input images are real images, the goal of the generator is to maximize D(x).

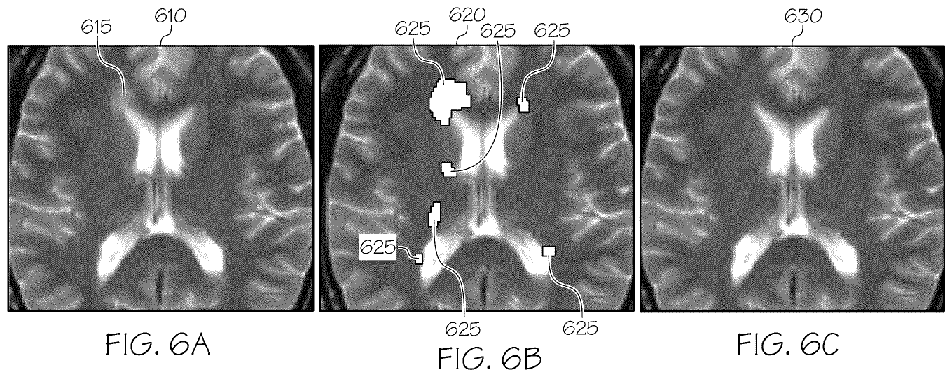

FIGS. 6A-C illustrate an exemplary transformation of original image 610 to an exemplary inpainting result 630 on an axial slice from a T2-weighted brain MRI scan. The original image 610 includes lesions 615, and these lesions 615 can be masked to form masked lesions image 620, including lesion masks 625. By inpainting the lesion masks 625, a synthetic lesion-free image 630 can be created.

An artificial neural network or an ensemble of such networks can be trained from multiple lesion-free slices from one of multiple MR multi-parametric images to synthetize partially missing healthy tissue imaging content. During the compilation and normalization of the training data set, the machine learning system may analyze the non-diseased portions of the MRI scans (e.g., the part of MRI scans showing white matter which is at least 2 mm away from any lesion mask) in order to be able to generate an approximation of what the lesion-free brain tissue may have looked like prior to the lesion formation. This approximation may then be inpainted into versions of the MRI scans that have had the masked regions of diseased lesion tissue removed. The resulting composite scan images (partially MR image and partially AI-generated) may approximate that which would be otherwise unavailable: a scan of the subject taken prior to the formation of lesions. Biomarker discovery may then be imposed to improve the symmetry of the data set, which in turn may provide improved separability between lesion types (e.g., acute versus chronic) and lesion progression status (e.g., chronic active or inactive, expanding/evolving or non-expanding/evolving, and/or harboring the specific pattern of a lesion subtype) in both lesion-free and lesion-present domains.

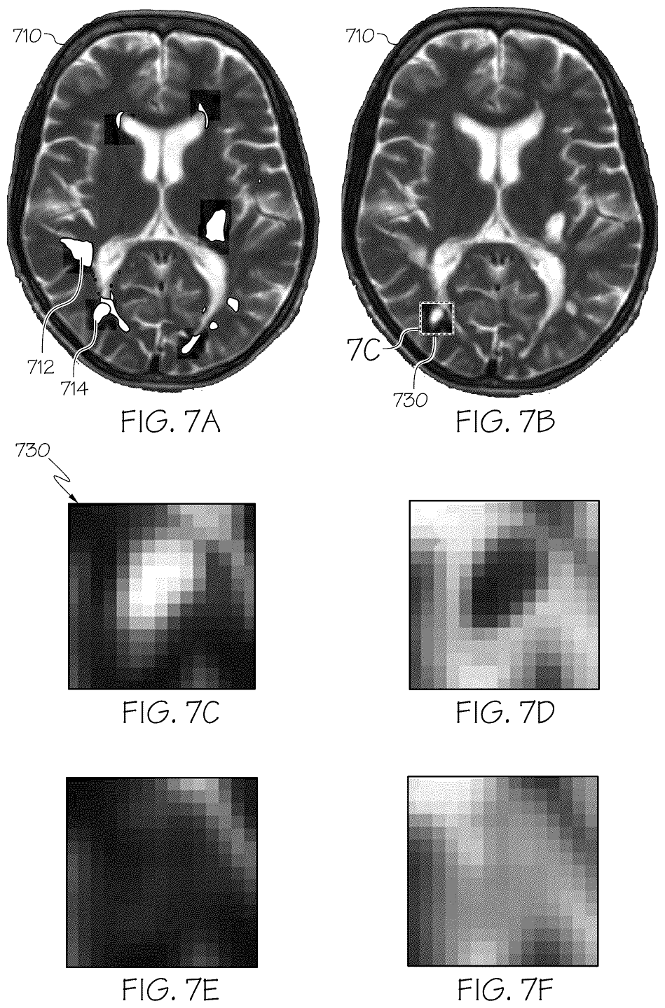

Returning to FIG. 2, at step 230, patches representing brain lesions in the first training data can be extracted. FIGS. 7A-F depict an exemplary patch sampling and extraction procedure. Acute and chronic segmentation map 710 can include acute lesions 712 and chronic lesions 714 on an axial view of a T2-weighted MRI. The masked lesions 712 and 714 may be referenced with respect to unmasked MRI image 720 to extract one or more patches 730. In the exemplary images 710 and 720, the central voxel of patch 730 is labeled as acute, and therefore patch 730 will be labeled as acute. In some embodiments, patch extraction may include inclusion/exclusion criteria. For example, patches relating to lesions that fail to meet inclusion criteria such as being smaller than a minimum size (e.g., <9 mm) or lesions that appear multi focal, may be excluded from the patches extracted for further analysis. These exclusion criteria may be designed to reduce the bias of the model to rely on lesion volume in its classifications, as the remaining patches can be of similar volume distribution.

The patches may be identified for extraction based on one or more of the imaging sequences, however, the patches can be extracted from any remaining images that correspond to the same physical space on other sequences. For example, FIG. 7D shows what a patch corresponding to patch 730 may look like in a corresponding T1-weighted MR image. These T2-weighted and T1-weighted images may then both be inpainted as illustrated in FIGS. 7E and 7F respectively.

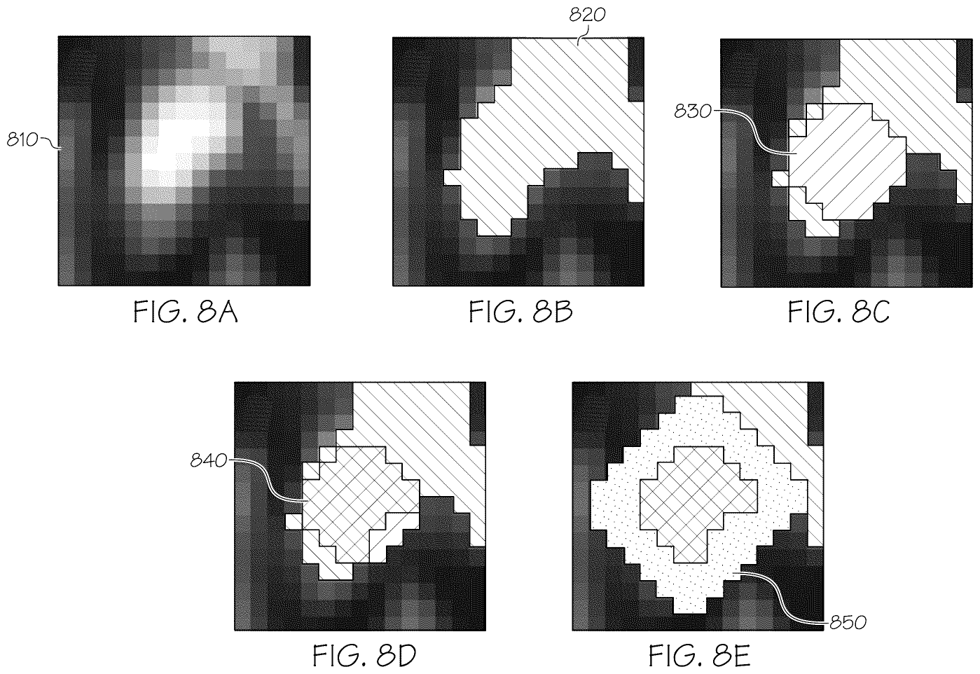

Conventionally, imaging biomarker feature extraction often relies on an exact delineation of the lesion masks defined by considering as ground truth the visual observation of lesion border limits as defined by the radiologist while extracting the source feature of the biomarker features by averaging measures over the totality of these masks treated as a single volume. In the context of confluent focal lesions forming a multifocal conglomerate of lesions, this can result in image information being concatenated across potentially different types of lesion foci. However, exemplary methods according to this disclosure can include a process for defining the relevant patches and segments of those patches (e.g., the core and periphery segments) automatically. FIGS. 8A-E illustrate an exemplary process for segmenting lesion patch 810.

After extraction, lesion patch 810 may have a lesion mask 820 applied to the entirety of the WMH region. Separately, different regions within the patch may be defined adaptively in relation to the lesion contained in the patch. Focus region 830 may be a binary ball containing the set of voxels located less than, for example, 4 mm away from the central voxel of the patch. Core region 840 may then be defined as the intersection of lesion mask 820 and focus region 830. Periphery region 850 may then be defined as the set of voxels located within, for example, 3 mm of the edge of core region 840, outside of core region 840. These regions, core region 840 and periphery region 850, are the regions within which biomarkers, including radiomic features, may be computed.

The partitions between the lesion subtype masks may be identified via an implicit lesion partition technique. The partition technique in accordance with the disclosure may employ, for example, two distinct categories of lesion features: (i) the core of the lesion that can correspond to the expected minimal volume of an acute lesion, and (ii) periphery of the lesion corresponding to a ring that follows the geometric properties of the lesion and captures inter-dependencies between healthy and diseased tissue. In some embodiments, the focus region can be approximately as large as the largest expected focal lesion size such that for all patches centered on focal lesions, the core region would fit the lesion mask and the patch-level classifier would be equivalent to a lesion-level approach. The periphery region may be defined as, for example, a ring of voxels located between about 4 mm and 7 mm away from the central voxel of the patch. Such a partition may allow capturing of the underlying pathological state of the lesion as well as evidence on the expansion/interaction with surrounding healthy tissue that is valuable information regarding its potential progression in time.

In some embodiments, a partition technique in accordance with the disclosure may employ additional categories, for example, three distinct categories of lesion features: (i) the core of the lesion that can correspond to the expected minimal volume of an acute lesion, (ii) the inner ring of the lesion (surrounding the core) that typically is part of the lesion and corresponds to a ring that follows the geometric properties of the lesion, and (iii) the periphery of the lesion that describes the features on the boundary of the inner ring of the lesion that captures inter-dependencies between healthy and diseased tissue. Such a partition allows capturing of the underlying pathological state of the lesion (core and inner ring) and provides evidence on the expansion/interaction with surrounding healthy tissue (outer ring) that is valuable information regarding its potential progression in time.

Once the lesions have been identified and patches have been extracted and segmented, the patches may be re-sampled for class-balancing purposes. Due to the training data likely including many more chronic patches than acute patches (patches are only considered acute for a limited time, but appear as chronic for a greater period of time), it may be beneficial to under-sample the chronic patches to reach an appropriate ratio for training. This re-sampling may include matching the samples by features such as lesion volume (e.g., class-balancing), to further limit bias in the trained model.

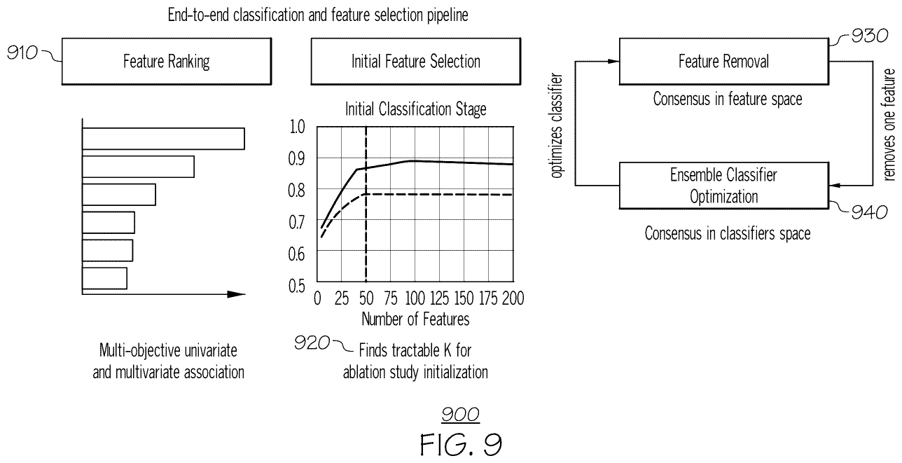

Referring again to FIG. 2, at step 240, biomarkers can be selected and extracted from the class-balanced collection of patches. FIG. 9 illustrates an exemplary classification and feature selection pipeline 900 that can evaluate the extent to which each biomarker is predictive of the appropriate classification (acute or chronic) for a given lesion. For example, in some embodiments, the first and second training data sets may be used, as the input to an ensemble classification method that seeks the optimal combination of machine learning methods and the optimal subset of features that could create the best possible separation on the reduced imaging biomarker space between lesion types (e.g., acute versus chronic lesions) or progression stage. In some embodiments, imaging biomarker selection pipeline 900 may use linear and non-linear feature-to-class correlation tests to identify the features that account for the highest variance between the classifications.

This evaluation and classification may employ initial feature ranking 910, and an initial feature selection 920 that may, for example, identify a number of features (e.g., 50 features) with the strongest individual correlation with the second training data set. From those features, embedded selection methods can leverage tree-based classifiers and linear models (e.g., boosted ensemble of trees, logistic regression, linear support vector machine). Then, starting from a feature subspace comprising a number (e.g., 50) of the most-relevant features, a recursive feature elimination process can be conducted, whereby the size of the feature subspace is recursively decremented by feature removal 930, which can eliminate the least useful feature at each recursive step. This recursive approach can cycle between ensemble classifier optimization 940, and feature removal 930 to arrive at a ranking where each decremented combination of biomarkers is associated with a prevalence that deciphers the importance of this feature space with respect to the lesion classification objective (e.g., acute versus chronic).

The outcome of this ensemble classification mechanism may be a selected subset of classification methods that may involve linear (e.g., logistic regression, support vector machines) and/or non-linear classifications methods (e.g., multi-layer perceptron, deep convolutional neural networks) acting on a low dimensional subset of imaging biomarkers that optimizes the separability between the two classes. Using that feature space, a pool of machine learning models may undergo hyperparameter tuning via an extensive grid search, which may be performed via a k-fold cross-validation on the classification task of interest. This tuning may then lead to a performance benchmark that can select the highest performing models, for example, the n top-performing models. These models may then be combined under a stacking or a winner takes all or a probabilistic importance sampling ensemble strategy.

As mentioned above, in some embodiments, the classifier may be further refined via a recursive feature elimination process. The recursive feature elimination process may reduce the number of required features by removing one feature at a time, re-running the ensemble classifier, and evaluating the relative impact of the removed feature. This iterative approach leads to a highly compact (i.e., reduced dimensionality) imaging biomarker signature on which the ensemble classification process is applied without sacrificing accuracy. And at step 250, a subset of the biomarkers can be determined based on the results of the recursive feature elimination process. In some embodiments, classification models developed and refined using methods disclosed herein have exhibited accuracy beyond 70% for both classifying acute lesions as being acute (e.g., 74.2%) and classifying chronic lesions as being chronic (e.g., 75.7%) in evaluations having over 2500 sample lesions.

In one aspect of the disclosure, a series of features have been found, via the above-discussed methods, to have predictive value with respect to the classification of a lesion, and in particular the classification of a brain lesion as either acute or chronic. For example, the following features have such predictive value.
Features may be identified that quantify the first order intensity of the core region of a lesion as it appears on a T2-weighted scan image. Such features account for acute lesions tending to be more intense than chronic lesions and more uniformly hyperintense, whereas chronic lesions may contain less hyperintense voxels.
Features may be identified that quantify the power of low-gray level signals around the periphery of a lesion as it appears on a T1-weighted scan image.
Features may be selected that quantify the power of high-gray level signals that are present in the periphery and/or core of a lesion as it appears on a T1-weighted scan image.
Features may be selected that relate to the inhomogeneity present in the images. For example, features may quantify the complexity of the image (the image is non-uniform and may include rapid changes in the gray levels), the variance of the gray levels with respect to a mean gray level, or the existence of homogenous patterns in the images.
Features may be selected that relate to the structure of the image, as relating to the presence of repeating patterns. For example, an image with more repeating patterns may be considered to be more “structured” than one with fewer observable intensity patterns.
Features may be selected that relate to the texture of the images, such as the coarseness or fineness of an image.

Returning to FIG. 1, at step 130, the classification model can generate a classification for each of the lesions identified in the MRI patient data, for example, classifying lesions as acute or chronic, gadolinium-enhancing or non-enhancing acute, chronic active or chronic inactive. At step 140, the generated classifications can then be provided for display or visualization so that a patient and/or care provider can review the classifications. In some embodiments, treatment plans may be generated for a patient based on the provided classifications. For example, some treatments can include guidelines regarding suitability or eligibility for use, and single point in time classification may allow treatments to be prescribed without the need to wait for detectable lesion development over time (e.g., 12-36 weeks). In some circumstances, the ability to begin a course of treatment weeks or months sooner than using conventional longitudinal scan information can have significant impacts on disease progression and/or symptom management.

With the method identifying the features having the most predictive value as it relates to the classification of a lesion, the resulting machine-learning based classifier may be able to accurately and reproducibly discriminate acute from chronic MS lesions using unenhanced T1-/T2-weighted information from a single MRI study.

As a result, the disclosed method may be able to effectively increase the sensitivity of a single time-point acute MS lesion detection, and may be able to replicate, approach, or exceed the sensitivity of traditional detection of hyperintensities identified on a T1-weighted scan with gadolinium enhancement and/or of new hyperintense lesions on a T2-weighted scan in comparison with a prior reference scan, which may be reflective of new local inflammation.

In a clinical context, a patient, such as one suspected of having a brain ailment such as multiple sclerosis, may be referred for an MRI scan of the brain at a single time point, and without agent contrast. The scan may then be input into the classifier algorithm. The classifier algorithm may then identify and distinguish between acute and chronic lesions present on the brain scan. Based on that identification and distinction, a healthcare practitioner may be able to prescribe treatment that is suitable to the particular patient and disease state. As the patient's treatment proceeds, additional scans may be conducted to monitor the efficacy of the treatment and the disease progression, however the classifier may significantly reduce the amount of scans with contrast and the need of a prior reference scan for the assessment of MS disease activity. For example, patients may change healthcare providers or otherwise lose access to prior scans, and single point in time classification can further reduce duplicative scans, and particularly scans with a paramagnetic contrast agent.

As discussed above, embodiments of this disclosure relate to analysis of MR images of MS patients to enable conclusions to be drawn that are not presently practical based on a radiologist's visual inspection alone. By collecting and analyzing sets of MRIs from MS patients taken both before and after the appearance of an acute MS lesion, the disclosed methods may be able to identify novel features within MRI images that precede lesion formation. These features may not currently be reliably detected using standard MRI analytical methods. An exemplary method 1000 of predicting lesion formation using a trained lesion prediction model is illustrated in FIG.

At step 1010, patient MRI data can be accessed. This patient data may be, for example, current data collected at a single point in time. At step 1020, the patient MRI data can then be provided as an input to a prediction model.

Building on the classification methods disclosed above, additional training on longitudinal T1- and T2-weighted MRI data (i.e., MRI scans of the same portion of a patient's anatomy at multiple points in time), may involve locating lesions on MRI scans and examining the precise regions of lesion formation in scans conducted prior to the lesion becoming detectable by traditional methods to build a lesion prediction model. By comparing these pre-lesion regions to a spatially matched patch from a single other patient and also to random patches in normal appearing tissue (e.g., normal-appearing white matter) of other patients, the disclosed methods may be able to identify and extract features that suggest future lesion formation. Exemplary methods may include identifying patches that have a detectable lesion, but that did not have a detectable lesion on a prior scan of the region of the patch. For example, the scans may be conducted 24-48 weeks apart. For these identified patches, a patch may be extracted from the same physical location in a brain scan of a different patient who did not have a detectable lesion in the patch location. This other patient may be monitored, and upon determining that no MS lesion appears within this patch for a period of time such as the next 24-48 weeks, the method is able to determine that the spatially matched patch from the other patient's scan may be used as a lesion-negative control patch. The lesion-positive patch, and the lesion negative patch from the other patient may then be used to train the classifier with control negatives alongside the known positives.

This model can, at step 1030, generate a prediction for the MRI patient data, for example, an indication of a likelihood of future lesion formation. At step 1040, the generated predictions can then be provided for display or visualization so that a patient and/or care provider can review the predictions. In some embodiments, treatment plans may be generated for a patient based on the provided predictions.

Methods according to the present disclosure may provide spatiotemporal predictions of the progression of a lesion (e.g., an acute MS lesion) in a manner that may be capable of guiding therapeutic strategies. The result of these methods may be otherwise unavailable or difficult to obtain information regarding the translation from healthy tissue to a lesion. Methods in accordance with the present disclosure may be capable of predicting the formation and progression of a lesion (e.g., an acute MS lesion) based on a single-time point MRI signal.

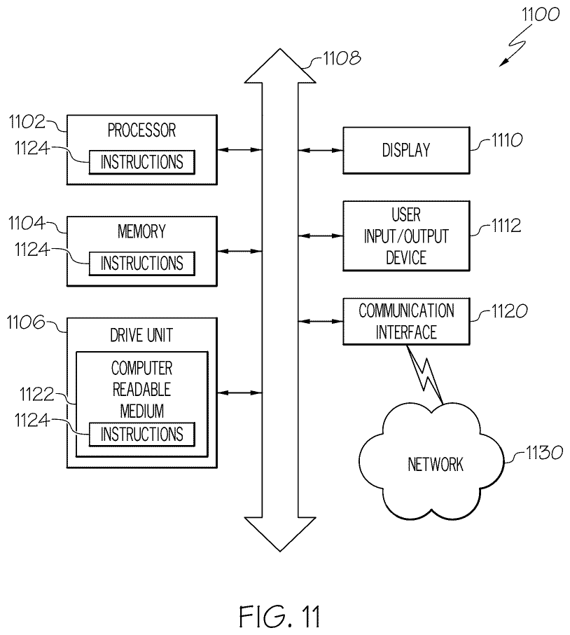

FIG. 11 is a simplified functional block diagram of a computer 1100 that may be configured as a device for executing the methods according to embodiments of the present disclosure. In various embodiments, any of the systems herein may be a computer 1100 including, for example, a data communication interface 1120 for packet data communication. The computer 1100 also may include a central processing unit (“CPU”) 1102, in the form of one or more processors, for executing program instructions. The computer 1100 may include an internal communication bus 1108, and a storage unit 1106 (such as ROM, HDD, SDD, etc.) that may store data on a computer readable medium 1122, although the computer 1100 may receive programming and data via network 1130. The computer 1100 may also have a memory 1104 (such as RAM) storing instructions 1124 for executing techniques presented herein, although the instructions 1124 may be stored temporarily or permanently within other modules of computer 1100 (e.g., processor 1102 and/or computer readable medium 1122). The computer 1100 also may include input and output ports 1112 and/or a display 1110 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

As used herein, a “machine learning model” is a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data. Unsupervised approaches may include clustering, or the like. K-means clustering or K-Nearest Neighbors may also be used, which may be supervised or unsupervised. Combinations of K-Nearest Neighbors and an unsupervised clustering technique may also be used. Any suitable type of training may be used, e.g., stochastic, gradient boosted, random seeded, recursive, epoch or batch-based, etc.

The general discussion of this disclosure provides a description of a suitable computing environment in which the present disclosure may be implemented. In one embodiment, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer.

Aspects of the present disclosure may be embodied in a general or special purpose computer and/or data processor that is specifically programmed, configured, and/or constructed to perform one or more computer-executable instructions for implementing the disclosed methods. While aspects of the present disclosure, such as certain functions, may be described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), Cloud Computing, and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only, with a true scope of the disclosure being indicated by the following claims and their equivalents.

What is claimed is:

1. A method of classifying brain lesions based on single point in time imaging, the method comprising:
accessing, by a system server, patient image data from a single point in time;
providing, by the system server, the patient image data as an input to a brain lesion classification model;
generating, by the brain lesion classification model, a classification for each of one or more lesions identified in the patient image data; and
providing the classification for each of the one or more lesions for display on one or more display devices;
wherein the brain lesion classification model is trained using subject image data for a plurality of subjects, the subject image data for each of the plurality of subjects being captured at two or more points in time.

2. The method of claim 1, wherein the patient image data from the single point in time includes data from two or more image scan sequences.

3. The method of claim 2, wherein the data from two or more image scan sequences include magnetic resonance imaging (MRI) data, and wherein the two or more image scan sequences do not include administration of paramagnetic contrast agents.

4. The method of claim 1, wherein the classification for each of one or more lesions identified in the patient image data is selected to be one of acute or chronic.

5. The method of claim 1, wherein the subject image data for the plurality of subjects is re-sampled to a common domain.

6. The method of claim 5, wherein the re-sampled subject image data for the plurality of subjects is bias-field corrected and normalized to have a zero mean and unit variance.

7. The method of claim 1, wherein the subject image data for the plurality of subjects includes synthetically generated inpainted data representing lesion free tissue.

8. The method of claim 1, wherein training the brain lesion classification model includes:
extracting, from the subject image data, one or more patches representing one or more brain lesions;
extracting, from each of the one or more patches representing one or more brain lesions, a plurality of biomarkers; and
identifying, within the plurality of biomarkers, a subset of biomarkers relevant to the classification of the one or more brain lesions.

9. The method of claim 8, wherein extracting one or more patches includes:
excluding one or more patches that fail to meet inclusion criteria related to a minimum lesion volume; and
segmenting one or more remaining patches into core and periphery regions.

10. A system, comprising:
a memory configured to store instructions; and
a processor operatively connected to the memory and configured to execute the instructions to perform a process for classifying brain lesions based on single point in time imaging, including:
accessing, by a system server, patient image data from a single point in time;
providing, by the system server, the patient image data as an input to a brain lesion classification model;
generating, by the brain lesion classification model, a classification for each of one or more lesions identified in the patient image data; and
providing the classification for each of the one or more lesions for display on one or more display devices;
wherein the brain lesion classification model is trained using subject image data for a plurality of subjects, the subject image data for each of the plurality of subjects being captured at two or more points in time.

11. The system of claim 10, wherein the patient image data from the single point in time includes data from two or more magnetic resonance imaging (MRI) scan sequences, and wherein the two or more MRI scan sequences do not include administration of paramagnetic contrast agents.

12. The system of claim 10, wherein the subject image data for the plurality of subjects is re-sampled to a common domain.

13. The system of claim 12, wherein the re-sampled subject image data for the plurality of subjects is bias-field corrected and normalized to have a zero mean and unit variance.

14. The system of claim 10, wherein the subject image data for the plurality of subjects includes synthetically generated inpainted data representing lesion free tissue.

15. The system of claim 10, wherein training the brain lesion classification model includes:
extracting, from the subject image data, one or more patches representing one or more brain lesions;
extracting, from each of the one or more patches representing one or more brain lesions, a plurality of biomarkers; and
identifying, within the plurality of biomarkers, a subset of biomarkers relevant to the classification of the one or more brain lesions.

16. The system of claim 15, wherein extracting one or more patches includes:
excluding one or more patches that fail to meet inclusion criteria related to a minimum lesion volume; and
segmenting one or more remaining patches into core and periphery regions.

17. A method for training a machine-learning model for classifying brain lesions, the method comprising:
obtaining, via a system server, first training data that includes information for a plurality of subjects including image scan data for each subject captured at two or more points in time;
obtaining, via the system server, second training data that includes classification information for one or more brain lesions present in the image scan data, wherein the classification information for the one or more brain lesions present in the image scan data is indicative of a classification of the one or more brain lesions as being acute or chronic;
extracting, from the first training data, one or more patches representing one or more brain lesions;
extracting, from each of the one or more patches representing one or more brain lesions, a plurality of biomarkers; and
determining, within the plurality of biomarkers, a subset of biomarkers relevant to the classification of the one or more brain lesions as correlated with the second training data.

18. The method of claim 17, wherein two or more points in time separated by at least about one week.

19. The method of claim 17, wherein the first training data includes synthetically generated inpainted data representing lesion free tissue.

20. A method of predicting a formation of brain lesions based on single point in time imaging, the method comprising:
accessing, by a system server, patient image data from a single point in time;
providing, by the system server, the patient image data as an input to a brain lesion prediction model;
generating, by the brain lesion prediction model, a prediction for the patient image data, the prediction including an indication of a likelihood of a future lesion forming; and
providing the prediction for the patient image data for display on one or more display devices;
wherein the brain lesion prediction model is trained using subject image data for a plurality of subjects, the subject image data for each of the plurality of subjects being captured at two or more points in time.
